SEQUENCE LISTING (54 sequences)

SEQ ID NO: 1 - protein, 112 aa, Artificial Sequence
  CD3 Z endodomain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 152 aa, Artificial Sequence
  CD28 and CD3 Zeta endodomains
SKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 188 aa, Artificial Sequence
  CD28, OX40 and CD3 Zeta endodomains
SKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 341 aa, Artificial Sequence
  CD148 endodomain
RKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 5 - protein, 707 aa, Artificial Sequence
  CD45 endodomain
KIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 6 - protein, 352 aa, Artificial Sequence
  SHP-1 phosphatase domain
FWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 7 - protein, 351 aa, Artificial Sequence
  SHP-2 phosphatase domain
FWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIVDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLVDQTSGDQSPLPPCTPTPPCAEMREDSARVYENVGLMQQQRSFR

SEQ ID NO: 8 - protein, 38 aa, Artificial Sequence
  ICOS endodomain
CWLTKKKYSSSVHDPNGEYMFMRAVNTAKKSRLTDVTL

SEQ ID NO: 9 - protein, 48 aa, Artificial Sequence
  CD27 endodomain
QRRKYRSNKGESPVEPAEPCHYSCPREEEGSTIPIQEDYRKPEPACSP

SEQ ID NO: 10 - protein, 111 aa, Artificial Sequence
  BTLA endodomain
RRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 11 - protein, 188 aa, Artificial Sequence
  CD30 endodomain
HRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 12 - protein, 58 aa, Artificial Sequence
  GITR endodomain
QLGLHIWQLRSQCMWPRETQLLLEVPPSTEDARSCQFPEEERGERSAEEKGRLGDLWV

SEQ ID NO: 13 - protein, 60 aa, Artificial Sequence
  HVEM endodomain
CVKRRKPRGDVVKVIVSVQRKRQEAEGEATVIEALQAPPDVTTVAVEETIPSFTGRSPNH

SEQ ID NO: 14 - protein, 97 aa, Artificial Sequence
  PD1 endodomain
CSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 15 - protein, 97 aa, Artificial Sequence; identical to SEQ ID NO: 14
  PDCD1 endodomain

SEQ ID NO: 16 - protein, 141 aa, Artificial Sequence
  BTLA4 endodomain
KLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 17 - protein, 168 aa, Artificial Sequence
  LILRB1 endodomain
LRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 18 - protein, 101 aa, Artificial Sequence
  LAIR1 endodomain
HRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 19 - protein, 62 aa, Artificial Sequence
  CTLA4 endodomain
FLLWILAAVSSGLFFYSFLLTAVSLSKMLKKRSPLTTGVYVKMPPTEPECEKQFQPYFIPIN

SEQ ID NO: 20 - protein, 111 aa, Artificial Sequence
  KIR2DL1 endodomain
GNSRHLHVLIGTSVVIIPFAILLFFLLHRWCANKKNAVVMDQEPAGNRTVNREDSDEQDPQEVTYTQLNHCVFTQRKITRPSQRPKTPPTDIIVYTELPNAESRSKVVSCP

SEQ ID NO: 21 - protein, 143 aa, Artificial Sequence
  KIR2DL4 endodomain
GIARHLHAVIRYSVAIILFTILPFFLLHRWCSKKKENAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 22 - protein, 143 aa, Artificial Sequence
  KIR2DL5 endodomain
TGIRRHLHILIGTSVAIILFIILFFFLLHCCCSNKKNAAVMDQEPAGDRTVNREDSDDQDPQEVTYAQLDHCVFTQTKITSPSQRPKTPPTDTTMYMELPNAKPRSLSPAHKHHSQALRGSSRETTALSQNRVASSHVPAAGI

SEQ ID NO: 23 - protein, 111 aa, Artificial Sequence
  KIR3DL1 endodomain
KDPRHLHILIGTSVVIILFILLLFFLLHLWCSNKKNAAVMDQEPAGNRTANSEDSDEQDPEEVTYAQLDHCVFTQRKITRPSQRPKTPPTDTILYTELPNAKPRSKVVSCP

SEQ ID NO: 24 - protein, 97 aa, Artificial Sequence
  KIR3DL3 endodomain
KDPGNSRHLHVLIGTSVVIIPFAILLFFLLHRWCANKKNAVVMDQEPAGNRTVNREDSDEQDPQEVTYAQLNHCVFTQRKITRPSQRPKTPPTDTSV

SEQ ID NO: 25 - protein, 256 aa, Artificial Sequence
  sequence of tyrosine kinase domain of CSK
LKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 26 - protein, 449 aa, Homo sapiens
  MISC_FEATURE: full length C-terminal Src kinase (CSK)
SAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 27 - protein, 7 aa, Artificial Sequence
  intracellular retention signal
NQPLLTD

SEQ ID NO: 28 - protein, 15 aa, Artificial Sequence
  intracellular retention signal
KYKSRRSFIDEKKMP

SEQ ID NO: 29 - protein, 6 aa, Artificial Sequence
  intracellular retention signal
DEKKMP

SEQ ID NO: 30 - protein, 20 aa, Foot-and-mouth disease virus
RAEGRGSLLTCGDVEENPGP

SEQ ID NO: 31 - protein, 20 aa, Artificial Sequence
  self-cleaving peptide sequence
QCTNYALLKLAGDVESNPGP

SEQ ID NO: 32 - protein, 20 aa, Mus musculus
METDTLLLWVLLLWVPGSTG

SEQ ID NO: 33 - protein, 19 aa, Artificial Sequence
  signal peptide (one amino acid deletion)
METDTLLWVLLLWVPGSTG

SEQ ID NO: 34 - protein, 18 aa, Artificial Sequence
  signal peptide (two amino acid deletion)
METDTLLWVLLLVPGSTG

SEQ ID NO: 35 - protein, 17 aa, Artificial Sequence
  signal peptide (three amino acid deletion)
METDTLLWVLLLPGSTG

SEQ ID NO: 36 - protein, 16 aa, Artificial Sequence
  signal peptide (five amino acid deletion)
METDTLLVLLLPGSTG

SEQ ID NO: 37 - protein, 183 aa, Artificial Sequence
  CD22(2Ig) membrane proximal Ig domains
PRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 38 - protein, 363 aa, Artificial Sequence
  CD148 transmembrane and endodomain portion, CD148TM-CD148endo
AVFGCIFGALVIVTVGGFIFWRKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIAS

SEQ ID NO: 39 - protein, 18 aa, Artificial Sequence
  Foot-and-Mouth disease virus (FMDV) 2A peptide
EGRGSLLTCGDVEENPGP

SEQ ID NO: 40 - protein, 478 aa, Artificial Sequence
  anti-CD19 2nd generation CAR with a CD28-Zeta endodomain
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIYIWAPLAGTCGVLLLSLVITLYCRKKRSRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 23 aa, Artificial Sequence
  Linker-V5 tag-Linker sequence, V5_tag
DSSGKPIPNPLLGLDSSGGGGSA

SEQ ID NO: 42 - protein, 6 aa, Artificial Sequence
  ITIM conserved sequence
  MISC_FEATURE (1)..(1): Xaa may be Ser, Ile, Val or Leu
  misc_feature (2)..(2): Xaa can be any naturally occurring amino acid
  misc_feature (4)..(5): Xaa can be any naturally occurring amino acid
  MISC_FEATURE (6)..(6): Xaa may be Ile, Val or Leu
XXYXXX

SEQ ID NO: 43 - protein, 4 aa, Artificial Sequence
  intracellular retention signal
  misc_feature (4)..(4): Xaa can be any naturally occurring amino acid
NPFX

SEQ ID NO: 44 - protein, 4 aa, Artificial Sequence
  intracellular retention signal
KDEL

SEQ ID NO: 45 - protein, 4 aa, Artificial Sequence
  intracellular retention signal
  misc_feature (3)..(4): Xaa can be any naturally occurring amino acid
KKXX

SEQ ID NO: 46 - protein, 5 aa, Artificial Sequence
  intracellular retention signal
  misc_feature (2)..(2): Xaa can be any naturally occurring amino acid
  misc_feature (4)..(5): Xaa can be any naturally occurring amino acid
KXKXX

SEQ ID NO: 47 - protein, 4 aa, Artificial Sequence
  intracellular retention signal sequence
  misc_feature (3)..(3): Xaa can be any naturally occurring amino acid
NPXY

SEQ ID NO: 48 - protein, 4 aa, Artificial Sequence
  intracellular retention signal sequence
  misc_feature (2)..(3): Xaa can be any naturally occurring amino acid
  MISC_FEATURE (4)..(4): Xaa is an amino acid with a bulky hydrophobic side chain
YXXX

SEQ ID NO: 49 - protein, 6 aa, Artificial Sequence
  intracellular retention signal sequence
  MISC_FEATURE (1)..(1): Xaa may be Asp or Glu
  misc_feature (2)..(4): Xaa can be any naturally occurring amino acid
  MISC_FEATURE (6)..(6): Xaa may be Leu or Ile
XXXXLX

SEQ ID NO: 50 - protein, 5 aa, Artificial Sequence
  intracellular retention signal sequence
  misc_feature (2)..(3): Xaa can be any naturally occurring amino acid
DXXLL

SEQ ID NO: 51 - protein, 5 aa, Artificial Sequence
  intracellular retention signal sequence
  misc_feature (2)..(2): Xaa can be any naturally occurring amino acid
  misc_feature (4)..(4): Xaa can be any naturally occurring amino acid
FXDXF

SEQ ID NO: 52 - protein, 5 aa, Artificial Sequence
  intracellular retention signal sequence
  MISC_FEATURE (2)..(2): Xaa is an amino acid with a bulky hydrophobic side chain
  misc_feature (3)..(3): Xaa can be any naturally occurring amino acid
  MISC_FEATURE (4)..(4): Xaa is an amino acid with a bulky hydrophobic side chain
  MISC_FEATURE (5)..(5): Xaa may be Asp or Glu
LXXXX

SEQ ID NO: 53 - protein, 5 aa, Artificial Sequence
  intracellular retention signal sequence
LLDLL

SEQ ID NO: 54 - protein, 5 aa, Artificial Sequence
  intracellular retention signal sequence
PWDLW

(19) United States
(12) Patent Application Publication
(10) Pub. No.: US 2020/0199550 A1
(43) Pub. Date: Jun. 25, 2020

(54) CELL COMPRISING A CHIMERIC ANTIGEN RECEPTOR (CAR)

(71) Applicant: AUTOLUS LIMITED, London (GB)

(72) Inventors: Shaun Cordoba, London (GB); Evangelia Kokalaki, London (GB); Martin Pulé, London (GB); Simon Thomas, London (GB); Shimobi Onuoha, London (GB)

(21) Appl. No.: 16/613,312

(22) PCT Filed: May 14, 2018
(86) PCT No.: PCT/GB18/51295

§ 371 Date: Nov. 13, 2019

(30) Foreign Application Priority Data
May 15, 2017 (GB) 1707783.5

(51) Int. Cl. C12N 9/16; C07K 14/725; C07K 16/28; C12N 15/85; C12N 5/0783; A61K 35/17

(52) U.S. Cl. CPC C12N 9/16; C07K 14/7051; C07K 16/2803; C12N 15/85; C07K 2319/04; A61K 35/17; C07K 2319/02; C07K 2319/03; C07K 2319/70; C12N 5/0636

(57) ABSTRACT

The present invention provides a cell which comprises; (i) a chimeric antigen receptor (CAR) which comprises an antigen binding domain and an intracellular signalling domain; and (isi) a membrane-tethered signal-dampening component (SDC) comprising a signai-dampening domain (SDD).

### FIELD OF THE INVENTION

The invention relates to a cell which comprises a chimeric antigen receptor (CAR).

### BACKGROUND TO THE INVENTION

A number of immunotherapeutic agents have been described for use in cancer treatment, including therapeutic monoclonal antibodies (mAbs), immunoconjugated mAbs, radioconjugated mAbs, bi-specific T-cell engagers and chimeric antigen receptors (CARS).

Chimeric antigen receptors are proteins which graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals (see FIG. 1A).

The most common form of these molecules are fusions of single-chain variable fragments (scFv) derived from monoclonal antibodies which recognize a target antigen, fused via a spacer and a trans-membrane domain to a signaling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

A problem with immunotherapeutic approaches targeting tumour associated antigens is that many tumour antigens are also expressed on normal tissue. The antigen her2, for example, is expressed at a low level in several normal tissues, including heart and pulmonary vasculature. PSMA is highly expressed in metastatic prostate cancer but is also detected in type II astrocytes, the renal proximal tubule, and the jejunum brush border. ROR1 is expressed in a subset of leukemias and lymphomas but is also detected in adipocytes.

Truly tumour-specific antigens are extremely rare and therefore most CARs are designed to redirect the T-cell towards an antigen that is merely overexpressed on a tumour. This results in a safety concern known as on-target toxicity where T-cells react to normal tissue expressing low doses of the target antigen.

In some cases, on-target, off-tumour responses can be managed by other means. For example, CD19-targeted CARs have been developed for the treatment of haematological malignancies because the resulting B-cell aplasia can be effectively managed by administering intravenous immunoglobulin. However, for many other cancers and target antigens, the collateral damage may not be manageable or tolerable. In a study investigating the treatment of metastatic renal cell carcinoma with CAIX-specific CAT-T cells, liver enzyme disturbances were observed due to expression of CAIX on the bile-duct epithelium. In a separate study, investigating the use of an ERBB2-specific CAR to treat colon cancer metastatic to the lungs and liver, respiratory distress was observed within 15 minutes of cell infusion, due to pulmonary infiltration. It is thought that the administered cells localised to the lung immediately following infusion and were triggered to release cytokine by the recognition of low levels of ERBB2 on lung epithelial cells.

There is therefore a need for alternative immunotherapeutic approaches which address the issue of on-target toxicity.

### DESCRIPTION OF THE FIGURES

FIG. 1—a) Schematic diagram illustrating a classical CAR. (b) to (d): Different generations and permutations of CAR endodomains: (b) initial designs transmitted ITAM signals alone through FcϵR1-γ or CD3ζ endodomain, while later designs transmitted additional (c) one or (d) two co-stimulatory signals in the same compound endodomain.

FIG. 2(a)—Diagram of immediate T-cell activation pathways. T-cell receptor activation results in phosphorylation of ITAMs. Phosphorylated ITAMs are recognized by the ZAP70 SH2 domains. Upon recognition, ZAP70 is recruited to the juxta-membrane region and its kinase domain subsequently phosphorylates LAT. Phosphorylated LAT is subsequently recognized by the SH2 domains of GRAP, GRB2 and PLC-γ. (b)—Diagram of immediate T-cell inhibition pathways. Activation of an inhibitory immune-receptor such as PD1 results in phosphorylation of ITIM domains. These are recognized by the SH2 domains of PTPN6. Upon recognition, PTPN6 is recruited to the juxta-membrane region and its phosphatase domain subsequently de-phosphorylates ITAM domains inhibiting immune activation.

FIG. 3—Schematic diagram of a dampened CAR system of the invention. The cell comprises a chimeric antigen receptor and a membrane-tethered signal dampening component. In this example, the signal dampening component comprises an ectodomain with two Ig domains from CD22, a transmembrane domain and the endodomain from CD148. CD148 endodomain dephosphorylates ITAMs in the intracellular signalling domain of the CAR and dampens CAR signalling.

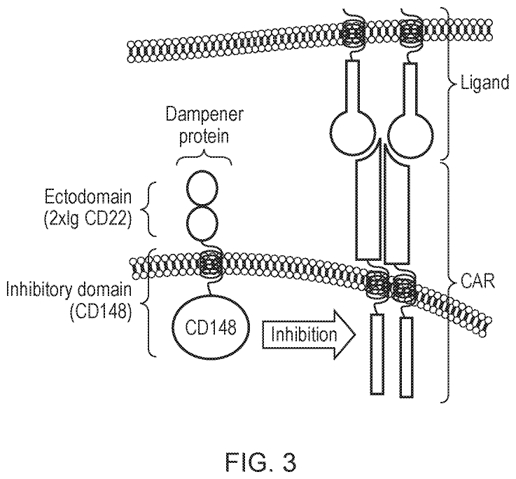

FIG. 4—Schematic diagram of a dampened CAR system of the invention. The cell comprises a chimeric antigen receptor and a membrane-tethered signal dampening component. In this example, the signal dampening component is tethered to the membrane using truncated Lck which acts as a membrane anchor. The signal dampening component comprises the endodomain from CD148 which dephosphorylates ITAMs in the intracellular signalling domain of the CAR and dampens CAR signalling.

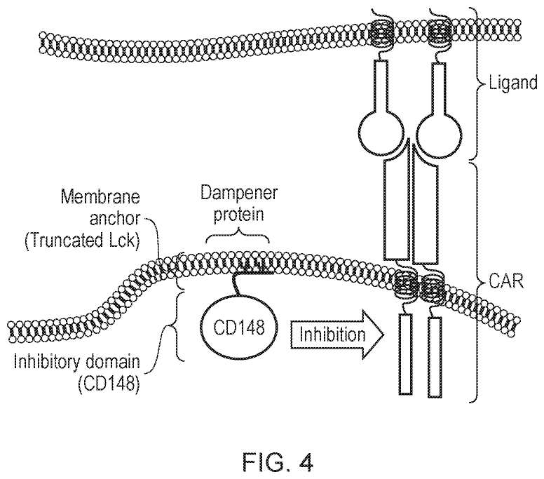

### SUMMARY OF ASPECTS OF THE INVENTION

The present inventors have developed a CAR-expressing cell which is capable of discriminating between cancerous and normal tissue based on the density of the target antigen. This is achieved by co-expressing a CAR with a phosphatase “damper” which causes dephosphorylation of the CAR endodomain, raising the threshold to activation.

Thus, in a first aspect, the present invention provides a cell which comprises;

(i) a chimeric antigen receptor (CAR) which comprises an antigen binding domain and an intracellular signalling domain; and

(iii) a membrane-tethered signal-dampening component (SDC) comprising a signal-dampening domain (SDD).

The SDD may be capable of inhibiting the intracellular signalling domain of the CAR.

The SDD may comprise a phosphatase domain capable of dephosphorylating immunoreceptor tyrosine-based activation motifs (ITAMs), for example the endodomain of CD148 or CD45 or the phosphatase domain of SHP-1 or SHP-2

The SDD may comprise an immunoreceptor tyrosine-based inhibition motif (ITIM), for example the SDD may comprise an endodomain from one of the following inhibitory receptors: PD1, BTLA, 2B4, CTLA-4, GP49B, Lair-1, Pir-B, PECAM-1, CD22, Siglec 7, Siglec 9, KLRG1, ILT2, CD94-NKG2A and CD5.

The SDD may inhibits a Src protein kinase, such as Lck. The SDD may comprise the kinase domain of CSK.

The membrane-tethered SDC may comprise a transmembrane domain or a myristoylation sequence.

The chimeric antigen receptor and/or the membrane-tethered signal-dampening component may comprise an intracellular retention sequence.

Both the CAR and the SDC may comprise a signal peptide and the signal peptide of the CAR may have a different amino acid sequence from the signal peptide of the SDC.

In a second aspect, the present invention provides a nucleic acid construct which comprises:
(i) a first nucleic acid sequence which encodes a chimeric antigen receptor (CAR) as defined in any preceding claim; and
(ii) a second nucleic acid sequence which encodes a membrane-tethered signal-dampening component (SDC) as defined in any preceding claim.

In a third aspect the present invention provides a kit of nucleic acid sequences comprising:
(i) a first nucleic acid sequence which encodes a chimeric antigen receptor (CAR) as defined herein;
(ii) a second nucleic acid sequence which encodes a membrane-tethered signal-dampening component (SDC) as defined herein.

In a fourth aspect there is provided a vector comprising a nucleic acid construct according to the second aspect of the invention.

In a fifth aspect there is provided kit of vectors which comprises:
(i) a first vector which comprises a nucleic acid sequence which encodes a chimeric antigen receptor (CAR) as defined herein;
(ii) a second vector which comprises a nucleic acid sequence which encodes a membrane-tethered signal-dampening component (SDC) as defined herein.

In a sixth aspect there is provided a pharmaceutical composition comprising a plurality of cells according to the first aspect of the invention.

In a seventh aspect, there is provided a pharmaceutical composition according to the sixth aspect of the invention for use in treating and/or preventing a disease.

In an eighth aspect there is provided a method for treating and/or preventing a disease, which comprises the step of administering a pharmaceutical composition according to the sixth aspect of the invention to a subject.

The method may comprise the following steps:
(i) isolation of a cell-containing sample;
(ii) transduction or transfection of the cells with a nucleic acid construct according to the second aspect of the invention, a kit of nucleic acid sequences according to the third aspect of the invention; a vector according to the fourth aspect of the invention or a kit of vectors according to the fifth aspect of the invention; and
(iii) administering the cells from (ii) to a subject.

In a ninth aspect, the present invention provides the use of a pharmaceutical composition according to the sixth aspect of the invention in the manufacture of a medicament for the treatment and/or prevention of a disease.

The disease may be cancer.

In a tenth aspect, there is provided a method for making a cell according to the first aspect of the invention, which comprises the step of introducing a nucleic acid construct according to the second aspect of the invention, a kit of nucleic acid sequences according to the third aspect of the invention; a vector according to the fourth aspect of the invention or a kit of vectors according to the fifth aspect of the invention into a cell.

The cell may be from a sample isolated from a subject.

The cell of the present invention is capable of discriminating between cancerous and normal tissue based on the density of the target antigen. The cell responds to antigen “dose” and is only activated to kill the cell when it expresses high levels of target antigen. This means that healthy tissue which express a low level of target antigen, for example lung epithelial cells expressing a low level of ERBB2 should be spared.

This opens up a whole new section of antigens as potential targets for CAR T cells. As explained in the background section, truly tumour-specific antigens are extremely rare. Many antigens are known to be expressed on tumours, but are also expressed at low levels on normal tissue. Engineering the T cell to discriminate between cancerous and normal tissue based on antigen dose is therefore extremely powerful because it makes it possible to target a wide spectrum of TAAs which were previously thought to be unsafe due to predicted problems of on-target off-tumour toxicity.

### DETAILED DESCRIPTION

Chimeric Antigen Receptors (CAR)

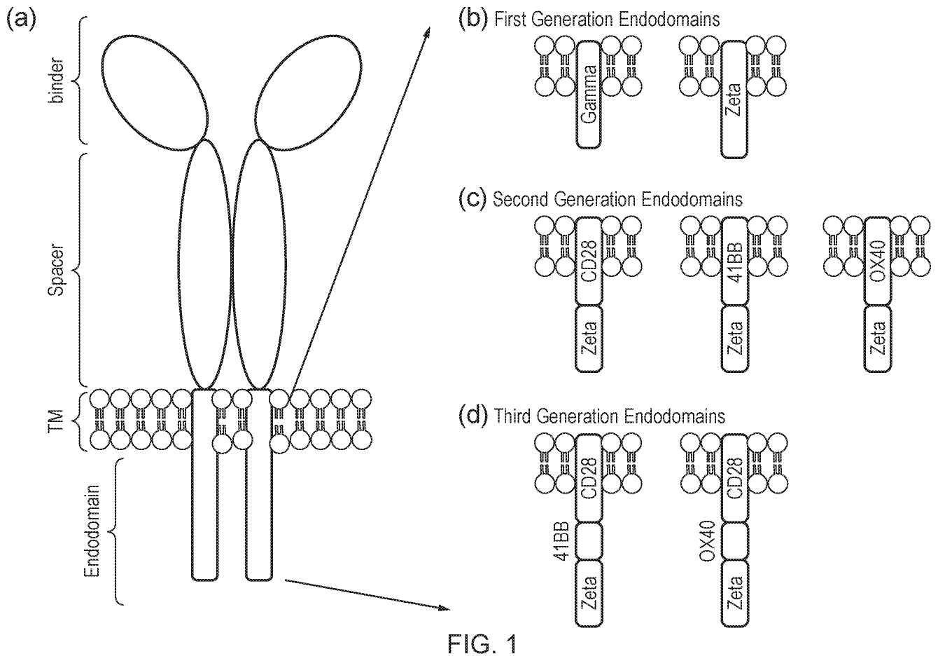

Classical CARs, which are shown schematically in FIG. 1, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site or on a ligand for the target antigen. A spacer domain may be necessary to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcϵR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral vectors. In this way, a large number of antigen-specific T cells can be generated for adoptive cell transfer. When the CAR binds the target-antigen, this results in the transmission of an activating signal to the T-cell it is expressed on. Thus the CAR directs the specificity and cytotoxicity of the T cell towards cells expressing the targeted antigen.

Antigen Binding Domain

The antigen-binding domain is the portion of a classical CAR which recognizes antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder single as a Darpin; or a single-chain derived from a T-cell receptor.

Various tumour associated antigens (TAR) are known, as shown in the following Table 1. The antigen-binding domain used in the present invention may be a domain which is capable of binding a TAA as indicated therein.

TABLE 1

| Cancer type | TAA |
|---|---|
| Diffuse Large B-cell Lymphoma | CD19, CD20 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| ultiple myeioma | BCMA, CD138 |
| Renal Cell Carcinoma | Carbonic anhydrase IX, G250 |
| Prostate cancer | PSMA |
| Bowel cancer | A33 |

The antigen-binding domain may comprise a proliferation-inducing ligand (APRIL) which binds to B-cell membrane antigen (BCMA) and transmembrane activator and calcium modulator and cyclophilin ligand interactor (TACI). A CAR comprising an APRIL-based antigen-binding domain is described in WO2015/052538.

Transmemebrane Domain

The transmembrane domain is the sequence of a classical CAR that spans the membrane. It may comprise a hydrophobic alpha helix. The transmembrane domain may be derived from CD28, which gives good receptor stability.

Signal Peptide

The CAR may comprise a signal peptide so that when it is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

Spacer Domain

The CAR may comprise a spacer sequence to connect the antigen-binding domain with the transmembrane domain. A flexible spacer allows the antigen-binding domain to orient in different directions to facilitate binding.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

Intracellular Signalling Domain

The intracellular signalling domain is the signal-transmission portion of a classical CAR.

The most commonly used signalling domain component is that of CD3-zeta endodomain, which contains 3 ITAMs. This transmits an activation signal to the T cell after antigen is bound. CD3-zeta may not provide a fully competent activation signal and additional co-stimulatory signalling may be needed. For example, chimeric CD28 and OX40 can be used with CD3-Zeta to transmit a proliferative/survival signal, or all three can be used together (illustrated in FIG. 1B).

The CAR may comprise the sequence shown as SEQ ID NO: 1, 2 or 3 or a variant thereof having at least 80% sequence identity.

SEQ ID NO: 1
CD3 Z endodomain
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR
SEQ ID NO: 2
CD28 and CD3 Zeta endodomains
SKRSRLLHSDYMNMTPRRPGPTRKHYQPYAPPRDFAAYRSRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PR
SEQ ID NO: 3
CD28, OX40 and CD3 Zeta endodomains
SKRSRLLHSDYMNMTPRRPGPTRKHYQPYAPPRDFAAYRSRDQRLPPDAH
KPPGGGSFRTPIQEEQADAHSTLAKIRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPR

A variant sequence may have at least 80%, 85%, 90%, 95%, 98% or 99% sequence identity to SEQ ID NO: 1, 2 or 3, provided that the sequence provides an effective intracellular signalling domain.

Signal Dampening Component (SDC)

The signal dampening component (SDC) is positioned at the intracellular side of the cell membrane, may it can exert its dampening effect on the intracellular signalling domain of the CAR.

The SDC may be tethered to the cell membrane, such that it acts as an anchor, tethering the signal dampening component to the intracellular surface of the cell membrane. In this respect, the SDC may comprise a membrane tethering component.

The membrane tethering component may comprise a membrane localisation domain. This may be any sequence which causes the signal dampening domain to be attached to or held in a position proximal to the plasma membrane.

It may be a sequence which causes the nascent polypeptide to be attached initially to the ER membrane. As membrane material “flows” from the ER to the Golgi and finally to the plasma membrane, the protein remain associated with the membrane at the end of the synthesis/translocation process.

The membrane localisation domain may, for example, comprise a transmembrane sequence, a stop transfer sequence, a GPI anchor or a myristoylation/prenylation/palmitoylation site.

Alternatively the membrane localisation domain may direct the SDC to a protein or other entity which is located at the cell membrane, for example by binding the membrane-proximal entity. The membrane tethering component may, for example, comprise a domain which binds a molecule which is involved in the immune synapse, such as TCR/CD3, CD4 or CD8.

Myristoylation is a lipidation modification where a myristoyl group, derived from myristic acid, is covalently attached by an amide bond to the alpha-amino group of an N-terminal glycine residue. Myristic acid is a 14-carbon saturated fatty acid also known as n-Tetradecanoic acid. The modification can be added either co-translationally or post-translationally. N-myristoyltransferase (NMT) catalyzes the myristic acid addition reaction in the cytoplasm of cells. Myristoylation causes membrane targeting of the protein to which it is attached, as the hydrophobic myristoyl group interacts with the phospholipids in the cell membrane.

The SDC of the cell of the present invention may comprise a sequence capable of being myristoylated by a NMT enzyme. For example, it may comprise a myristoyl group when expressed in a cell.

The membrane tethering component may comprise a consensus sequence such as: NH2-G1-X2-X3-X4-S5-X6-X7-X8 which is recognised by NMT enzymes.

Palmitoylation is the covalent attachment of fatty acids, such as palmitic acid, to cysteine and less frequently to serine and threonine residues of proteins. Palmitoylation enhances the hydrophobicity of proteins and can be used to induce membrane association. In contrast to prenylation and myristoylation, palmitoylation is usually reversible (because the bond between palmitic acid and protein is often a thioester bond). The reverse reaction is catalysed by palmitoyl protein thioesterases.

In signal transduction via G protein, palmitoylation of the a subunit, prenylation of the γ subunit, and myristoylation is involved in tethering the G protein to the inner surface of the plasma membrane so that the G protein can interact with its receptor.

The SDC may comprise a sequence capable of being palmitoylated. For example, it may comprise additional fatty acids when expressed in a cell which causes membrane localisation.

Prenylation (also known as isoprenylation or lipidation) is the addition of hydrophobic molecules to a protein or chemical compound. Prenyl groups (3-methyl-but-2-en-1-yl) facilitate attachment to cell membranes, similar to lipid anchors like the GPI anchor.

Protein prenylation involves the transfer of either a farnesyl or a geranyl-geranyl to moiety to C-terminal cysteine(s) of the target protein. There are three enzymes that carry out prenylation in the cell, farnesyl transferase, Caax protease and geranylgeranyl transferase I.

The SDC may comprise a sequence capable of being prenylated. For example, it may comprise one or more prenyl groups when expressed in a cell which causes membrane localisation.

Signal Dampening Domain

The signal-dampening component (SDC) of the cell of the present invention also comprises a signal-dampening domain (SDD).

The signal-dampening domain inhibits CAR-mediated cell signalling.

The signal dampening domain may inhibit CAR-mediated cell signalling completely, or it may cause partial inhibition, effectively “turning down” CAR-mediated cell signalling.

The signal dampening domain may result in signalling through the signalling component which is 2, 5, 10, 50, 100, 1,000 or 10,000-fold lower than the signalling which occurs in the absence of the signal dampening domain.

CAR mediated signalling may be determined by a variety of methods known in the art. Such methods include assaying signal transduction, for example assaying levels of specific protein tyrosine kinases (PTKs), breakdown of phosphatidylinositol 4,5-biphosphate (PIP2), activation of protein kinase C (PKC) and elevation of intracellular calcium ion concentration. Functional readouts, such as clonal expansion of T cells, upregulation of activation markers on the cell surface, differentiation into effector cells and induction of cytotoxicity or cytokine (e.g. IL-2) secretion may also be utilised.

Control of T Cell Signalling

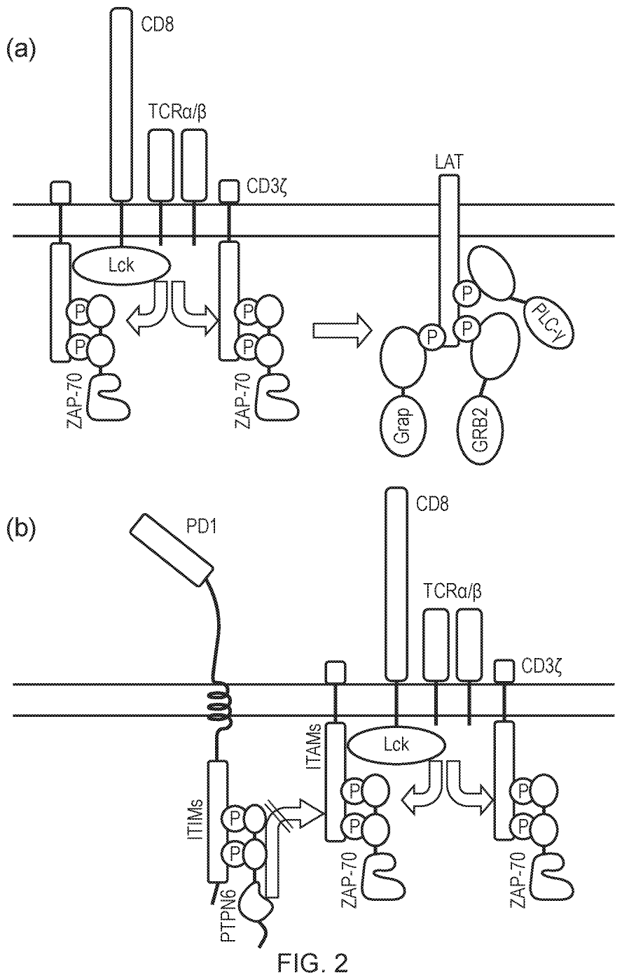

The earliest step in T cell activation is the recognition of a peptide MHC-complex on the target cell by the TCR. This initial event causes the close association of Lck kinase with the cytoplasmic tail of CD3-zeta in the TCR complex. Lck then phosphorylates immunoreceptor tyrosine-based activation motifs (ITAMs) in the cytoplasmic tail of CD3-zeta which allows the recruitment of ZAP70. ZAP70 is an SH2 containing kinase that plays a pivotal role in T cell activation following engagement of the TCR. Tandem SH2 domains in ZAP70 bind to the phosphorylated CD3 resulting in ZAP70 being phosphorylated and activated by Lck or by other ZAP70 molecules in trans. Active ZAP70 is then able to phosphorylate downstream membrane proteins, key among them the linker of activated T cells (LAT) protein. LAT is a scaffold protein and its phosphorylation on multiple residues allows it to interact with several other SH2 domain-containing proteins including Grb2, PLC-g and Grap which recognize the phosphorylated peptides in LAT and transmit the T cell activation signal downstream ultimately resulting in a range of T cell responses. This process is summarized in FIG. 2A.

T cell activation is controlled by kinetic segregation at the T-cell:target cell synapse. At the ground state, the signalling components on the T-cell membrane are in dynamic homeostasis whereby dephosphorylated ITAMs are favoured over phosphorylated ITAMs. This is due to greater activity of the transmembrane CD45/CD148 phosphatases over membrane-tethered kinases such as Ick. When a T-cell engages a target cell through a T-cell receptor (or CAR) recognition of cognate antigen, tight immunological synapses form. This close juxtapositioning of the T-cell and target membranes excludes CD45/CD148 due to their large ectodomains which cannot fit into the synapse. Segregation of a high concentration of T-cell receptor associated ITAMs and kinases in the synapse, in the absence of phosphatases, leads to a state whereby phosphorylated ITAMs are favoured. ZAP70 recognizes a threshold of phosphorylated ITAMs and propagates a T-cell activation signal.

In vivo, membrane-bound immunoinhibitory receptors such as CTLA4, PD-1, LAG-3, 2B4 or BTLA 1 also inhibit T cell activation. As illustrated schematically in FIG. 2B, inhibitory immune-receptors such as PD1 effectively reverse the first steps of the T-cell activation process. PD1 has ITIMs in its endodomain which are recognized by the SH2 domains of SHP-1 or SHP-2. Upon recognition, SHP-1 and/or SHP-2 is recruited to the juxta-membrane region and its phosphatase domain subsequently de-phosphorylates ITAM domains inhibiting immune activation.

Phosphatases

The signal dampening domain of the signal dampening component may comprise a phosphatase, such as a phosphatase capable of dephosphorylating an ITAM.

The signal dampening domain of the signal dampening component may comprise all of part of a receptor-like tyrosine phosphatase. The phospatase may interfere with the phosphorylation and/or function of elements involved in T-cell signalling, such as PLCγ1 and/or LAT.

The signal dampening domain may comprise the phosphatase domain of one or more phosphatases which are involved in controlling T-cell activation, such as CD148, CD45, SHP-1 or SHP-2.

CD148

CD148 is a receptor-like protein tyrosine phosphatase which negatively regulates TCR signaling by interfering with the phosphorylation and function of PLCγ1 and LAT.

The endodomain of CD148 is shown as SEQ ID No. 4.

CD148 endodomain sequence
SEQ ID No 4
RKKRKDAKNNEVSFSQIKPKKSKLIRVENFEAYFKKQQADSNCGFAEEY
EDLKLVGISQPKYAAELAENRGKNRYNNVLPYDISRVKLSVQTHSTDDY
INANYMPGYHSKKDFIATQGPLPNTLKDFWRMVWEKNVYAIIMLTKCVE
QGRTKCEEYWPSKQAQDYGDITVAMTSEIVLPEWTIRDFTVKNIQTSES
HPLRQFHFTSWPDHGVPDTTDLLINFRYLVRDYMKQSPPESPILVHCSA
GVGRTGTFIAIDRLIYQIENENTVDVYGIVYDLRMHRPLMVQTEDQYVF
LNQCVLDIVRSQKDSKVDLIYQNTTAMTIYENLAPVTTFGKTNGYIA

CD45

CD45 present on all hematopoetic cells, is a protein tyrosine phosphatase which is capable of regulating signal transduction and functional responses, again by phosphorylating PLC γ1.

The endodomain of CD45 is shown as SEQ ID No. 5.

CD45 endodomain sequence
SEQ ID 5
KIYDLHKKRSCNLDEQQELVERDDEKQLMNVEPIHADILLETYKRKIAD
EGRLFLAEFQSIPRVFSKFPIKEARKPFNQNKNRYVDILPYDYNRVELS
EINGDAGSNYINASYIDGFKEPRKYIAAQGPRDETVDDFWRMIWEQKAT
VIVMVTRCEEGNRNKCAEYWPSMEEGTRAFGDVVVKINQHKRCPDYIIQ
KLNIVNKKEKATGREVTHIQFTSWPDHGVPEDPHLLLKLRRRVNAFSNF
FSGPIVVHCSAGVGRTGTYIGIDAMLEGLEAENKVDVYGYVVKLRRQRC
LMVQVEAQYILIHQALVEYNQFGETEVNLSELHPYLHNMKKRDPPSEPS
PLEAEFQRLPSYRSWRTQHIGNQEENKSKNRNSNVIPYDYNRVPLKHEL
EMSKESEHDSDESSDDDSDSEEPSKYINASFIMSYWKPEVMIAAQGPLK
ETIGDFWQMIFQRKVKVIVMLTELKHGDQEICAQYWGEGKQTYGDIEVD
LKDTDKSSTYTLRVFELRHSKRKDSRTVYQYQYTNWSVEQLPAEPKELI
SMIQVVKQKLPQKNSSEGNKHHKSTPLLIHCRDGSQQTGIFCALLNLLE
SAETEEWDIFQVVKALRKARPGMVSTFEQYQFLYDVIASTYPAQNGQVK
KNNHQEDKIEFDNEVDKVKQDANCVNPLGAPEKLPEAKEQAEGSEPTSG
TEGPEHSVNGPASPALNQGS

SHP1/SHP2

Src homology region 2 domain-containing phosphatase-1 (SHP-1, also known as PTPN6) is a member of the protein tyrosine phosphatase family.

The N-terminal region of SHP-1 contains two tandem SH2 domains which mediate the interaction of PTPN6 and its substrates. The C-terminal region contains a tyrosine-protein phosphatase domain.

SHP-1 is capable of binding to, and propagating signals from, a number of inhibitory immune receptors or ITIM containing receptors, such as, PD1, PDCD1, BTLA4, LILRB1, LAIR1, CTLA4, KIR2DL1, KIR2DL4, KIR2DL5, KIR3DL1 and KIR3DL3.

Human SHP-1 protein has the UniProtKB accession number P29350.

The protein tyrosine phosphatase (PTP) domain of SHP-1 is shown below as sequence ID No. 6.

SHP-1 phosphatase domain
(SEQ ID NO: 6)
FWEEFESLQKQEVKNLHQRLEGQRPENKGKNRYKNILPFDHSRVILQGR
DSNIPGSDYINANYIKNQLLGPDENAKTYIASQGCLEATVNDFWQMAWQ
ENSRVIVMTTREVEKGRNKCVPYWPEVGNIQRAYGPYSVTNCGEHDTTE
YKLRTLQVSPLDNGDLIREIWHYQYLSWPDHGVPSEPGGVLSFLDQINQ
RQESLPHAGPIIVHCSAGIGRTGTIIVIDMLMENISTKGLDCDIDIQKT
IQMVRAQRSGMVQTEAQYKFIYVAIAQFIETTKKKLEVLQSQKGQESEY
GNITYPPAMKNAHAKASRTSSKHKEDVYENLHTKNKREEKVKKQRSADK
EKSKGSLKRK

SHP-2

SHP-2, also known as PTPN11, PTP-1D and PTP-2C is a member of the protein tyrosine phosphatase (PTP) family. Like PTPN6, SHP-2 has a domain structure that consists of two tandem SH2 domains in its N-terminus followed by a protein tyrosine phosphatase (PTP) domain. In the inactive state, the N-terminal SH2 domain binds the PTP domain and blocks access of potential substrates to the active site. Thus, SHP-2 is auto-inhibited. Upon binding to target phospho-tyrosyl residues, the N-terminal SH2 domain is released from the PTP domain, catalytically activating the enzyme by relieving the auto-inhibition.

Human SHP-2 has the UniProtKB accession number P35235-1.

The protein tyrosine phosphatase (PTP) domain of SHP-2 is shown below as sequence ID No. 7.

SHP-2 phosphatase domain
(SEQ ID NO: 7)
FWEEFETLQQQECKLLYSRKEGQRQENKNKNRYKNILPFDHTRVVLHDG
DPNEPVSDYINANIIMPEFETKCNNSKPKKSYIATQGCLQNTVNDFWRM
VFQENSRVIVMTTKEVERGKSKCVKYWPDEVALKEYGVMRVRNVKESAA
HDYTLRELKLSKVGQALLQGNTERTVWQYHFRTWPDHGVPSDPGGVLDF
LEEVHHKQESIVDAGPVVVHCSAGIGRTGTFIVIDILIDIIREKGVDCD
IDVPKTIQMVRSQRSGMVQTEAQYRFIYMAVQHYIETLQRRIEEEQKSK
RKGHEYTNIKYSLVDQTSGDQSPLPPCTPTPPCAEMREDSARVYENVGL
MQQQRSFR

The signal dampening domain may comprise the phosphatase domain of SEQ ID No 4, 5, 6 or 7 or a variant thereof. The variant may, for example, have at least 80, 85, 90, 95, 98 or 99% sequence identity, provided that the variant sequence is capable of dampening CAR-mediated cell signalling. Th variant phosphatase may be capable of dephosphorylating one or more ITAM(s).

Endodomains from Immunoregulatory Molecules

The signal dampening domain of the signal dampening component may comprise all or part of the endodomain of an immunoregulatory molecule which inhibits T cell signalling. For example, the signal dampening domain may comprise the endodomain from an immunoinhibitory receptor which inhibits T cell activation. The inhibitory receptor may be a member of the CD28 or Siglec family such as CTLA4, PD-1, LAG-3, 2B4, BTLA 1, CD28, ICOS. CD33, CD31, CD27, CD30, GITR or HVEM or Siglec-5, 6, 7, 8, 9, 10 or 11.

The signal dampening domain may comprise one or more immunoreceptor tyrosine-based inhibition motifs (ITIMs).

An ITIM is a conserved sequence of amino acids (S/I/V/LxYxxI/V/L) that is found in the cytoplasmic tails of many inhibitory receptors of the immune system. After ITIM-possessing inhibitory receptors interact with their ligand, their ITIM motif becomes phosphorylated by enzymes of the Src kinases.

Immune inhibitory receptors such as PD1, PDCD1, BTLA4, LILRB1, LAIR1, CTLA4, the Killer inhibitory receptor family (KIR) including KIR2DL1, KIR2DL4, KIR2DL5, KIR3DL1 and KIR3DL3 contain ITIMs.

The signal dampening domain may comprise one or more of the sequence(s) shown as SEQ ID NO: 8 to 24.

ICOS endodomain
SEQ ID NO: 8
CWLIKKKYSSSVHDPNGEYMFMRAVNTAKKSRLTDVTL
CD27 endodomain
SEQ ID NO: 9
QRRKYRSNKGESPVEPAEPCHYSCPREEEGSTIPQEDYRKPEPACSP
BTLA endodomain
SEQ ID NO: 10
RRHQGKQNELSDTAGREINLVDAHLKSEQTEASTRQNSQVLLSETGIYD
NDPDLCFRMQEGSEVYSNPCLEENKPGIVYASLNHSVIGPNSRLARNVK
EAPTEYASICVRS
CD30 endodomain
SEQ ID NO: 11
HRRACRKRIRQKLHLCYPVQTSQPKLELVDSRPRRSSTQLRSGASVTEP
VAEERGLMSQPLMETCHSVGAAYLESLPLQDASPAGGPSSPRDLPEPRV
STEHTNNKIEKIYIMKADTVIVGTVKAELPEGRGLAGPAEPELEEELEA
DHTPHYPEQETEPPLGSCSDVMLSVEEEGKEDPLPTAASGK
GITR endodomain
SEQ ID NO: 12
QLGLHIWQLRSQCMWPRETQLLLEVPPSTEDARSCQFPEEERGERSAEE
KGRLGDLWV
HVEM endodomain
SEQ ID NO: 13
CVKRRKPRGDVVKVIVSVQRKRQEAEGEATVIEALQAPPDVTTVAVEET
IPSFTGRSPNH
PD1 endodomain
SEQ ID No. 14
CSRAARGTIGARRTGQPLKEDPSAVPVFSVDYGELDFQWREKTPEPPVP
CVPEQTEYATIVFPSGMGTSSPARRGSADGPRSAQPLRPEDGHCSWPL
PDCD1 endodomain
SEQ ID No. 15
CSRAARGTIGARRTGQPLKEDPSAVPVFSVDYGELDFQWREKTPEPPVP
CVPEQTEYATIVFPSGMGTSSPARRGSADGPRSAQPLRPEDGHCSWPL
BTLA4 endodomain
SEQ ID 16
KLQRRWKRTQSQQGLQENSSGQSFFVRNKKVRRAPLSEGPHSLGCYNPM
MEDGISYTTLRFPEMNIPRTGDAESSEMQRPPPDCDDTVTYSALHKRQV
GDYENVIPDFPEDEGIHYSELIQFGVGERPQAQENVDYVILKH
LILRB1 endodomain
SEQ ID 17
LRHRRQGKHWTSTQRKADFQHPAGAVGPEPTDRGLQWRSSPAADAQEEN
LYAAVKHTQPEDGVEMDTRSPHDEDPQAVTYAEVKHSRPRREMASPPSP
LSGEFLDTKDRQAEEDRQMDTEAAASEAPQDVTYAQLHSLTLRREATEP
PPSQEGPSPAVPSIYATLAIH
LAIR1 endodomain
SEQ ID 18
HRQNQIKQGPPRSKDEEQKPQQRPDLAVDVLERTADKATVNGLPEKDRE
TDTSALAAGSSQEVTYAQLDHWALTQRTARAVSPQSTKPMAESITYAAV
ARH
CTLA4 endodomain
SEQ ID 19
FLLWILAAVSSGLFFYSFLLTAVSLSKMLKKRSPLTTGVYVKMPPTEPE
CEKQFQPYFIPIN
KIR2DL1 endodomain
SEQ ID 20
GNSRHLHVLIGTSVVIIPFAILLFFLLHRWCANKKNAVVMDQEPAGNRT
VNREDSDEQDPQEVTYTQLNHCVFTQRKITRPSQRPKTPPTDIIVYTEL
PNAESRSKVVSCP
KIR2DL4 endodomain
SEQ ID 21
GIARHLHAVIRYSVAIILFTILPFFLLHRWCSKKKENAAVMNQEPAGHR
TVNREDSDEQDPQEVTYAQLDHCIFTQRKITGPSQRSKRPSTDTSVCIE
LPNAEPRALSPAHEHHSQALMGSSRETTALSQTQLASSNVPAAGI
KIR2DL5 endodomain
SEQ ID 22
TGIRRHLHILIGTSVAIILFIILFFFLLHCCCSNKKNAAVMDQEPAGDR
TVNREDSDDQDPQEVTYAQLDHCVFTQTKITSPSQRPKTPPTDTTMYME
LPNAKPRSLSPAHKHHSQALRGSSRETTALSQNRVASSHVPAAGI
KIR3DL1 endodomain
SEQ ID 23
KDPRHLHILIGTSVVIILFILLLFFLLHLWCSNKKNAAVMDQEPAGNRT
ANSEDSDEQDPEEVTYAQLDHCVFTQRKITRPSQRPKTPPTDTILYTEL
PNAKPRSKVVSCP
KIR3DL3 endodomain
SEQ ID 24
KDPGNSRHLHVLIGTSVVIIPFAILLFFLLHRWCANKKNAVVMDQEPAG
NRTVNREDSDEQDPQEVTYAQLNHCVFTQRKITRPSQRPKTPPTDTSV

The signal dampening domain may comprise a variant of one of the sequences shown as SEQ ID NO: 8 to 24 having at least 80%, 85%, 90%, 95%, 98% or 99% sequence identity. The variant sequence may be able to recruit SHP-1 and/or SHP-2 to the cell membrane. The variant sequence may comprise one or more ITIM(s).

CSK Endodomain

Tyrosine-protein kinase CSK (C-terminal Src kinase) is an enzyme (UniProt ID: P41240 [http://www.uniprot.org/uniprot/P41240]) which phosphorylates tyrosine residues located in the C-terminal end of Src-family kinases (SFKs). The signal dampening domain may comprise the tyrosine kinase domain of CSK (SEQ ID No. 25) or just the tyrosine kinase domain (SEQ ID No. 26).

sequence of tyrosine kinase domain of CSK
SEQ ID No: 25
LKLLQTIGKGEFGDVMLGDYRGNKVAVKCIKNDATAQAFLAEASVMTQL
RHSNLVQLLGVIVEEKGGLYIVTEYMAKGSLVDYLRSRGRSVLGGDCLL
KFSLDVCEAMEYLEGNNFVHRDLAARNVLVSEDNVAKVSDFGLTKEASS
TQDTGKLPVKWTAPEALREKKFSTKSDVWSFGILLWEIYSFGRVPYPRI
PLKDVVPRVEKGYKMDAPDGCPPAVYEVMKNCWHLDAAMRPSFLQLREQ
LEHIKTHELHL
sequence of full length CSK
SEQ ID No: 26
SAIQAAWPSGTECIAKYNFHGTAEQDLPFCKGDVLTIVAVTKDPNWYKA
KNKVGREGIIPANYVQKREGVKAGTKLSLMPWFHGKITREQAERLLYPP
ETGLFLVRESTNYPGDYTLCVSCDGKVEHYRIMYHASKLSIDEEVYFEN
LMQLVEHYTSDADGLCTRLIKPKVMEGTVAAQDEFYRSGWALNMKELKL
LQTIGKGEFGDVMLGDYRGNKVAVKCIKNDATAQAFLAEASVMTQLRHS
NLVQLLGVIVEEKGGLYIVTEYMAKGSLVDYLRSRGRSVLGGDCLLKFS
LDVCEAMEYLEGNNFVHRDLAARNVLVSEDNVAKVSDFGLTKEASSTQD
TGKLPVKWTAPEALREKKFSTKSDVWSFGILLWEIYSFGRVPYPRIPLK
DVVPRVEKGYKMDAPDGCPPAVYEVMKNCWHLDAAMRPSFLQLREQLEH
IKTHELHL

The signal dampening domain may comprise a variant of the sequence or part thereof having at least 80% sequence identity, as long as the variant retains the capacity to inhibit T cell signalling.

Controlling Relative Expression of CAR and SDC

In the cell of the present invention, the signal-dampening component dephosphorylates the endodomain of the CAR, raising the threshold to activation. By altering the ratio of CAR to damper, it is possible to “tune” the threshold of CAR activation, for example such that the CAR-expressing cell is activated by a tumour cell expressing a high level of target antigen, but is not activated by a normal cell expressing a low level of target antigen.

It is possible to alter the ratio of expression of two proteins in a cell by various mechanisms known in the art.

For example, WO2016/174408 describes the use of an intracellular retention signal to modulate the relative expression of two polypeptides.

In the cell of the present invention, the CAR and/or the SDC may comprise an intracellular retention signal.

The intracellular retention signal may direct the transmembrane protein away from the secretory pathway and/or to a membrane-bound intracellular compartment such as a lysozomal, endosomal or Golgi compartment.

The intracellular retention signal may, for example, be a tyrosine-based sorting signal, a dileucine-based sorting signal, an acidic cluster signal, a lysosomal avoidance signal, an NPFX′(1,2)D-Type signal, a KDEL, a KKX′X′ or a KX′KX′X′ signal (wherein X′ is any amino acid).

The intracellular retention signal may comprise a sequence selected from the group of: NPX′Y, YX′X′Z′, [DE]X′X′X′L[LI], DX′X′LL, DP[FW], FX′DX′F, NPF, LZX′Z[DE], LLDLL, PWDLW, KDEL, KKX′X′ or KX′KX′X′; wherein X′ is any amino acid and Z′ is an amino acid with a bulky hydrophobic side chain.

The intracellular retention signal may comprise any of the sequences shown in Tables 1 to 5 of WO2016/174408.

The intracellular retention signal may comprise the Tyrosinase-related protein (TYRP)-1 intracellular retention signal. The intracellular retention signal may comprise the TYRP-1 intracellular domain. The intracellular retention signal may comprise the sequence NQPLLTD (SEQ ID No. 27).

The intracellular retention signal may comprise the Adenoviral E3/19K intracellular retention signal. The intracellular retention signal may comprise the E3/19K cytosolic domain. The intracellular retention signal may comprise the sequence KYKSRRSFIDEKKMP (SEQ ID No. 28); or DEKKMP (SEQ ID No. 29).

WO2016/174409 describes the use of altered signal peptides to modulate the relative expression of two polypeptides.

In the cell of the present invention, both the CAR and the SDC may comprise a signal peptide and the signal peptide of the CAR may have a different amino acid sequence from the signal peptide of the SDC.

One signal peptide may have fewer hydrophobic amino acids than the other signal peptide.

The signal peptide of the CAR and the signal peptide of the SDC may be derivable from the same sequence, but one signal peptide may comprise one or more amino acid deletions or substitutions to remove or replace one or more hydrophobic amino acids compared to the other signal peptide.

Signal sequences have a tripartite structure, consisting of a hydrophobic core region (h-region) flanked by an n- and c-region. The signal peptide of the CAR and the signal peptide of the SDC may have identical n- and c-regions, but may differ in the h-region: the h-region of one signal peptide having more hydrophobic amino acids that the other signal peptide.

Hydrophobic amino acids include: Alanine (A); Valine (V); Isoleucine (I); Leucine (L); Methionine (M); Phenylalanine (P); Tyrosine (Y); Tryptophan (W)—in particular: Valine (V); Isoleucine (I); Leucine (L); and Tryptophan (W).

The signal peptide of one polypeptide may comprise up to five more hydrophobic amino acids than the other signal peptide. The altered signal peptide may have up to 10%, up to 20%, up to 30%, up to 40% or up to 50% of its hydrophobic amino acids replaced or removed.

The present invention also provides a method for altering the threshold for activation of a cell according to the first aspect of the invention by altering the relative expression of the CAR and the SDC.

The relative expression of the CAR and the SDC may be altered, for example, by including one or more intracellular retention sequence(s) in the CAR and/or the SDC; or by altering the signal peptide of the CAR and/or the signal peptide of the SDC.

Nucleic Acid Construct

The present invention provides nucleic acid sequences encoding a chimeric antigen receptor (CAR); and/or a signal-dampening component (SDC) as defined above.

A nucleic acid sequence encoding the CAR may have the following structure:

AgB-spacer-TM-endo

in which

AgB is a nucleic acid sequence encoding an antigen-binding domain;

spacer is a nucleic acid sequence encoding a spacer;

TM1 is a nucleic acid sequence encoding a transmembrane domain;

endo is a nucleic acid sequence encoding an intracellular signalling domain.

A nucleic acid encoding the signal dampening component may have the following structure:

MLD-SDD; or

SDD-MLD

in which

MLD is a nucleic acid sequence encoding a membrane localisation domain; and

SDD is a nucleic acid sequence encoding a signal dampening domain

The present invention provides a nucleic acid construct which comprises:
(i) a first nucleic acid sequence which encodes a chimeric antigen receptor (CAR);
(ii) a second nucleic acid sequence which encodes a signal-dampening component (SDC).

The first and second nucleic acid sequences may be in either order in the construct.

In the construct, the nucleic acid sequences may be connected by sequences enabling co-expression of the CAR and SDC as separate polypeptides. For example, the nucleic acid may encode a cleavage site between the two components. The cleavage site may be self-cleaving, such that when the compound polypeptide is produced, it is immediately cleaved into the separate components without the need for any external cleavage activity.

Various self-cleaving sites are known, including the Foot-and-Mouth disease virus (FMDV) 2a self-cleaving peptide, which has the sequence shown:

SEQ ID NO: 30
RAEGRGSLLTCGDVEENPGP,
or
SEQ ID NO: 31
QCTNYALLKLAGDVESNPGP

The co-expressing sequence may be an internal ribosome entry sequence (IRES). The co-expressing sequence may be an internal promoter.

The nucleic acid construct may, for example, encode a polypeptide having the following structure:

SP1.V5_tag-CD22(2Ig)-CD148TM-CD148endo-2A-SP2-CAR

in which:

“SP1” is a signal peptide derived from murine Ig kappa chain V-III region. The wild type sequence has the sequence shown as SEQ ID No. 32. Suboptimal versions of this sequence may be used to alter the SDC:CAR protein ratio, for example as shown in Table 1. In the sequences shown in Table 1, hydrophobic residues are highlighted in bold. One or more of these residues are removed in the variant sequences. The effect of sequential removal of hydrophobic amino acids in a signal peptide on relative protein expression is described in the Examples of WO2016/174409.

(SEQ ID No. 32)
METDTLLLWVLLLWVPGSTG

| | | Seq |
|---|---|---|
| | | ID |
| | Sequence | No. |
| Wild-type sequence | METDTLLLWVLLLWVPGSTG | 32 |
| One amino acid dele- | METDTLLWVLLLWVPGSTG | 33 |
| tion | | |
| Two amino acid dele- | METDTLLWVLLLVPGSTG | 34 |
| tion | | |
| Three amino acid | METDTLLWVLLLPGSTG | 35 |
| deletion | | |
| Five amino acid | METDTLLVLLLPGSTG | 36 |
| deletion | | |

“V5_tag” is a Linker-V5 tag-Linker sequence having the sequence:

(SEQ ID No. 41)
DSSGKPIPNPLLGLDSSGGGGSA

“CD22(2Ig)” is the two most membrane proximal Ig domains from human CD22, having the sequence:

(SEQ ID No. 37)
PRDVRVRKIKPLSEIHSGNSVSLQCDFSSSHPKEVQFFWEKNGRLLGKE
SQLNFDSISPEDAGSYSCWVNNSIGQTASKAWTLEVLYAPRRLRVSMSP
GDQVMEGKSATLTCESDANPPVSHYTWFDWNNQSLPYHSQKLRLEPVKV
QHSGAYWCQGTNSVGKGRSPLSTLTVYYSPETIGRR

“CD148TM-CD148endo” is the transmembrane and endodomain portion from CD148 having the sequence:

(SEQ ID No. 38)
AVFGCIFGALVIVTVGGFIFWRKKRKDAKNNEVSFSQIKPKKSKLIRVE
NFEAYFKKQQADSNCGFAEEYEDLKLVGISQPKYAAELAENRGKNRYNN
VLPYDISRVKLSVQTHSTDDYINANYMPGYHSKKDFIATQGPLPNTLKD
FWRMVWEKNVYAIIMLTKCVEQGRTKCEEYWPSKQAQDYGDITVAMTSE
IVLPEWTIRDFTVKNIQTSESHPLRQFHFTSWPDHGVPDTTDLLINFRY
LVRDYMKQSPPESPILVHCSAGVGRTGTFIAIDRLIYQIENENTVDVYG
IVYDLRMHRPLMVQTEDQYVFLNQCVLDIVRSQKDSKVDLIYQNTTAMT
IYENLAPVTTFGKTNGYIAS

“2A” is an FMDV 2A self-cleaving peptide having the sequence:

(SEQ ID No. 39)
EGRGSLLTCGDVEENPGP

“SP2” is a signal peptide derived from murine Ig kappa chain V-III region, which may be the same or different from SP1. It may comprise the wild-type sequence (SEQ ID No. 32) or a suboptimal sequence with one or more deletions of hydrophobic amino acids (SEQ ID No 33 to 36)

“ CAR” is an anti-CD19 2nd generation CAR with a CD28-Zeta endodomain. The CAR having the sequence:

(SEQ ID No. 40)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEITKAGGGGSGGGGSGGGGSGGGGSEVKLQESGPGLVAPSQSL
SVTCTVSGVSLPDYGVSWIRQPPRKGLEWLGVIWGSETTYYNSALKSRL
TIIKDNSKSQVFLKMNSLQTDDTAIYYCAKHYYYGGSYAMDYWGQGTSV
TVSSDPTTTPAPRPPTPAPTIASQPLSLRPEACRPAAGGAVHTRGLDFA
CDIYIWAPLAGTCGVLLLSLVITLYCRKKRSRSKRSRLLHSDYMNMTPR
RPGPTRKHYQPYAPPRDFAAYRSRVKFSRSADAPAYQQGQNQLYNELNL
GRREEYDVLDKRRGRDPEMGGKPRRKNPQEGLYNELQKDKMAEAYSEIG
MKGERRRGKGHDGLYQGLSTATKDTYDALHMQALPPR.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

The present invention also provides a kit comprising a first nucleic acid sequences encoding a chimeric antigen receptor (CAR); a second nucleic acid sequence encoding a signal-dampening component (SDC).

Vector

The present invention also provides a vector, or kit of vectors which comprises one or more nucleic acid sequence(s) of the invention. Such a vector may be used to introduce the nucleic acid sequence(s) into a host cell so that it expresses the CAR and/or SDC as defined above.

The vector may, for example, be a plasmid or a viral vector, such as a retroviral vector or a lentiviral vector, or a transposon based vector or synthetic mRNA.

The vector may be capable of transfecting or transducing a T cell or a NK cell.

Cell

The present invention relates to a cell which comprises a dampenable CAR system.

The cell may comprise a nucleic acid or a vector of the present invention.

The cell may be an immune cell, such as a cytolytic immune cell. Cytolytic immune cells can be T cells or T lymphocytes which are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

Natural Killer Cells (or NK cells) are a type of cytolytic cell which form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CAR-expressing cells of the invention may be any of the cell types mentioned above.

CAR-expressing cells, such as T or NK cells may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, CAR—expressing cells may be derived from ex vivo differentiation of inducible progenitor cells or embryonic progenitor cells to T cells. Alternatively, an immortalized T-cell line which retains its lytic function and could act as a therapeutic may be used.

In all these embodiments, CAR cells are generated by introducing DNA or RNA coding for the receptor component and signalling component by one of many means including transduction with a viral vector, transfection with DNA or RNA.

The CAR cell of the invention may be an ex vivo T or NK cell from a subject. The T or NK cell may be from a peripheral blood mononuclear cell (PBMC) sample. T or NK cells may be activated and/or expanded prior to being transduced with nucleic acid encoding the molecules providing the CAR system according to the first aspect of the invention, for example by treatment with an anti-CD3 monoclonal antibody.

The cell of the invention may be made by:
(i) isolation of a cell-containing sample from a subject or other sources listed above; and
(ii) transduction or transfection of the cells with one or more a nucleic acid sequence(s) or nucleic acid construct as defined above.

The cells may then by purified, for example, selected on the basis of expression of the antigen-binding domain of the antigen-binding polypeptide.

Pharmaceutical Composition

The present invention also relates to a pharmaceutical composition containing a plurality of cells of the invention. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The present invention provides a method for treating and/or preventing a disease which comprises the step of administering the cells of the present invention (for example in a pharmaceutical composition as described above) to a subject.

A method for treating a disease relates to the therapeutic use of the cells of the present invention. In this respect, the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. In this respect, the cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a cell-containing sample;
(ii) transducing or transfecting such cells with a nucleic acid sequence or vector provided by the present invention;
(iii) administering the cells from (ii) to a subject.

The present invention provides a cell of the present invention for use in treating and/or preventing a disease.

The invention also relates to the use of a cell of the present invention in the manufacture of a medicament for the treatment and/or prevention of a disease.

The disease to be treated and/or prevented by the methods of the present invention may be an infection, such as a viral infection.

The methods of the invention may also be for the control of pathogenic immune responses, for example in autoimmune diseases, allergies and graft-vs-host rejection.

The methods may be for the treatment of a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The CAR cells of the present invention may be capable of killing target cells, such as cancer cells. The target cell may be recognisable by expression of a TAA, for example the expression of a TAA provided above in Table 1.

The CAR of the cell of the invention may recognise a target antigen which is expressed at a relatively high level on a malignant cell, but which is expressed at a relatively low level on one or more normal tissues.

The CAR may, for example, be specific for EGFR, ErbB2, GD2 or CAIX.

The invention will now be further described by way of Examples, which are meant to serve to assist one of ordinary skill in the art in carrying out the invention and are not intended in any way to limit the scope of the invention.

### EXAMPLES

### Example 1

### Creation of a Panel of Dampened CAR Constructs

A panel of bicistronic constructs are created, each having the having the general structure:

SP1.V5_tag-CD22(2Ig)-CD148TM-CD148endo-2A-SP2-CAR

When expressed, the transcript self-cleaves at the 2A site to produce a signal dampening component; with a CD148 phosphatase endodomain; and an anti-CD19 second generation CAR.

SP1 is the signal peptide of the SDC, whereas SP2 is the signal peptide of the CAR.

As described in WO2016/174409 it is possible to alter the ratios of expression of two transmembrane proteins by altering the sequences of their signal peptides.

In this study, both SP1 and SP2 are derived from murine Ig kappa chain V-III region. The wild type sequence has the sequence shown as SEQ ID No. 32. In order to test whether suboptimal versions of this sequence may be used to alter the SDC:CAR protein ratio, a panel of constructs were created in which hydrophobic amino acid sequences were deleted in a step-wise fashion from the signal peptides of the CAR or the SDC (Table 2). In the sequences shown in Table 2, hydrophobic residues are highlighted in bold.

TABLE 2

| Con- | SDC signal | CAR signal |
|---|---|---|
| struct | peptide sequence | peptide sequence |
| SDC/ | METDTLLLWVLLLWVPGSTG | METDTLLLWVLLLWVPGSTG |
| CAR | (SEQ ID No. 32) | (SEQ ID No. 32) |
| SDC-1/ | METDTLLWVLLLWVPGSTG | METDTLLLWVLLLWVPGSTG |
| CAR | (SEQ ID No. 33) | (SEQ ID No. 32) |
| SDC-2/ | METDTLLWVLLLVPGSTG | METDTLLLWVLLLWVPGSTG |
| CAR | (SEQ ID No. 34) | (SEQ ID No. 32) |
| SDC-3/ | METDTLLWVLLLPGSTG | METDTLLLWVLLLWVPGSTG |
| CAR | (SEQ ID No. 35) | (SEQ ID No. 32) |
| SDC-5/ | METDTLLVLLLPGSTG | METDTLLLWVLLLWVPGSTG |
| CAR | (SEQ ID No. 36) | (SEQ ID No. 32) |
| SDC/ | METDTLLLWVLLLWVPGSTG | METDTLLWVLLLWVPGSTG |
| CAR-1 | (SEQ ID No. 32) | (SEQ ID No. 33) |
| SDC/ | METDTLLLWVLLLWVPGSTG | METDTLLWVLLLVPGSTG |
| CAR-2 | (SEQ ID No. 32) | (SEQ ID No. 34) |
| SDC/ | METDTLLLWVLLLWVPGSTG | METDTLLWVLLLPGSTG |
| CAR-3 | (SEQ ID No. 32) | (SEQ ID No. 35) |
| SDC/ | METDTLLLWVLLLWVPGSTG | METDTLLVLLLPGSTG |
| CAR-5 | (SEQ ID No. 32) | (SEQ ID No. 36) |

The constructs are transiently transfected into 293T cells. Three days after transfection the 293T cells are stained with both (i) soluble chimeric CD19 fused with rabbit Fc chain, followed by anti-Rabbit Fc-FITC to detect the CAR; and (iii) and an anti-CD22 antibody to detect expression of the SDC. The cells are analysed by flow cytometry as a comparison with non-transfected (NT) cells.

### Example 2

### Testing the Panel of Construct in a Killing Assay

The panel of constructs described in Example 1 is expressed in BW5 cells, SupT1 cells (which are CD19 negative), are engineered to be CD19 positive giving target negative and positive cell lines which are as similar as possible. Primary human T-cells from 3 donors are transduced with: (i) “Classical” anti-CD19 CAR; and (ii) the panel of bi-cistronic “dampened” CD19 CAR system described in Table 2 above. Non-transduced T-cells and T-cells transduced with the different CAR constructs are challenged 1:1 with either SupT1 cells or SupT1.CD19 cells. Supernatant is sampled 48 hours after challenge. Supernatant from background (T-cells alone), and maximum (T-cells stimulated with PMA/Ionomycin) is also sampled. Interferon-gamma is measured in supernatants by ELISA.

Killing of target cells is also demonstrated using a chromium release assay. SupT1 and SupT1.CD19 cells are loaded with ⁵¹Cr and incubated with control and CAR T-cells. Lysis of target cells is determined by counting ⁵¹Cr in the supernatant.

### Example 3

### Testing the Panel of Constructs Against Target Cells with Different Levels of Antigen Expression

SupT1 target cells were created which express varying levels of the antigen CD19. This was achieved by expressing CD19 with a tyrp1 retention signal and varying the length of the linker between the transmembrane domain and the retention signal. Use of the tyrp1 retention signal to alter the expression level of a transmembrane protein is described in WO2016/174408.

SupT1 cells were created with very low, low, mid, and high expression of CD19, as shown in the following table. For the very low expressers, a double retention motif was used.

| | | Norm # of |
|---|---|---|
| | MFI | molecules |
| High | 59,902 | 628,687 |
| Very low | 250 | 1,745 |
| Mid | 2,111 | 21,304 |
| Low | 905 | 8,629 |
| NT | 84 | 0 |

The panel of constructs described in Example 1 are tested against the target cells with varying levels of antigen expression using the killing assays described in Example 2.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A cell which comprises;
(i) a chimeric antigen receptor (CAR) which comprises an antigen binding domain and an intracellular signalling domain; and
(iii) a membrane-tethered signal-dampening component (SDC) comprising a signal-dampening domain (SDD).

2. A cell according to claim 1, wherein the SDD inhibits the intracellular signalling domain of the CAR.

3. A cell according to claim 2, wherein the SDD comprises a phosphatase domain capable of dephosphorylating immunoreceptor tyrosine-based activation motifs (ITAMs).

4. A cell according to claim 3, wherein the SDD comprises the endodomain of CD148 or CD45.

5. A cell according to claim 3, wherein the SDD comprises the phosphatase domain of SHP-1 or SHP-2

6. A cell according to claim 2, wherein the SDD comprises an immunoreceptor tyrosine-based inhibition motif (ITIM).

7. (canceled)

8. A cell according to claim 2, wherein the SDD inhibits a Src protein kinase.

9-10. (canceled)

11. A cell according to claim 1, wherein the membrane-tethered SDC comprises a transmembrane domain or a myristoylation sequence.

12. A cell according to claim 1 wherein the chimeric antigen receptor and/or the membrane-tethered signal-dampening component comprise(s) an intracellular retention sequence.

13. A cell according to claim 1 wherein both the CAR and the SDC comprise a signal peptide and the signal peptide of the CAR has a different amino acid sequence from the signal peptide of the SDC.

14. A nucleic acid construct which comprises:
(i) a first nucleic acid sequence which encodes a chimeric antigen receptor (CAR) which comprises an antigen binding domain and an intracellular signaling domain; and
(ii) a second nucleic acid sequence which encodes a membrane-tethered signal-dampening component (SDC) comprising a signal-dampening domain (SDD).

15. A kit comprising:
(i) a first nucleic acid sequence or first vector which encodes a chimeric antigen receptor (CAR) which comprises an antigen binding domain and an intracellular signaling domain;
(ii) a second nucleic acid sequence or second vector which encodes a membrane-tethered signal-dampening component (SDC) comprising a signal-dampening domain (SDD).

16. A vector comprising a nucleic acid construct according to claim 14.

17. (canceled)

18. A pharmaceutical composition comprising a plurality of cells according to claim 1.

19. (canceled)

20. A method for treating and/or preventing a disease, which comprises the step of administering a pharmaceutical composition according to claim 18 to a subject.

21. A method according to claim 20, which comprises the following steps:
(i) isolation of a cell-containing sample;
(ii) transduction or transfection of the cells with a nucleic acid construct according to claim 14, a kit of nucleic acid sequences according to claim 15; a vector according to claim 16 or a kit of vectors according to claim 17; and
(iii) administering the cells from (ii) to a subject.

22. (canceled)

23. A method according to claim 20 wherein the disease is cancer.

24. A method for making a cell according to claim 1, which comprises the step of introducing into a cell:
(i) a first nucleic acid sequence which encodes a chimeric antigen receptor (CAR) as defined in any preceding claim which comprises an antigen binding domain and an intracellular signaling domain; and
(ii) a second nucleic acid sequence which encodes a membrane-tethered signal-dampening component (SDC) as defined in any preceding claim comprising a signal-dampening domain (SDD).

25. A method according to claim 24 wherein the cell is from a sample isolated from a subject.
